(19) United States
(12) Patent Application Publication
(10) Pub. No.: US 2026/0002948 A1
(43) Pub. Date: Jan. 1, 2026

(54) REAGENT FOR DETECTING METHYL SALICYLATE, METHYL SALICYLATE SENSOR, METHOD FOR SENSING METHYL SALICYLATE USING THE SAME, AND METHOD FOR DETECTING PLANT PATHOGEN INFECTION

(71) Applicant: NEC Corporation, Tokyo (JP)

(72) Inventor: Katsumi MAEDA, Tokyo (JP)

(73) Assignee: NEC Corporation, Tokyo (JP)

(21) Appl. No.: 19/237,218

(22) Filed: Jun. 13, 2025

(30) Foreign Application Priority Data
Jun. 27, 2024 (JP) 2024-103669

(51) Int. Cl. G01N 33/74; G01N 21/64

(52) U.S. Cl. CPC G01N 33/74; G01N 21/6428; G01N 2333/415

(57) ABSTRACT

The present invention provides a reagent for detecting methyl salicylate serving as a plant hormone released when the plant is infected with a pathogen, a methyl salicylate sensor, a method for sensing methyl salicylate using the same, and a method for detecting plant pathogen infection by the same in cultivation of plants including agricultural products. A reagent for detecting methyl salicylate, containing a terbium compound, and an amine. A methyl salicylate sensor including a capture unit including the reagent and used for capturing methyl salicylate, and a detection unit detecting whether methyl salicylate is captured by the capture unit.

This application is based upon and claims the benefit of priority from Japanese patent application No. 2024-103669, filed on Jun. 27, 2024, the disclosure of which is incorporated herein in its entirety by reference.

### TECHNICAL FIELD

The present disclosure relates to a reagent for detecting methyl salicylate serving as a plant hormone released when the plant is infected with a pathogen, the reagent containing a terbium compound and an amine, a methyl salicylate sensor, a method for sensing methyl salicylate using the same, and a method for detecting plant pathogen infection.

### BACKGROUND ART

It is known that when a plant is infected with a pathogen, the plant synthesizes and releases plant hormones that are signaling molecules, and informs surrounding plants of the pathogen infection, leading to promote a defense mechanism in advance. Quick recognizing such signaling molecules released by the plant enables detection of pest and disease damage.

As a method for detecting the pest damage in a target plant, for example, WO 2019/082942 A1 discloses a method of using a phenomenon in which a monitor plant containing a photoprotein gene receives, when arranged in the vicinity of a target plant, volatile substances released from the target plant in response to a stress, thereby emitting light.

### CITATION LIST

### Patent Literature

Patent Literature 1: WO2019/082942

### SUMMARY

An objective of the present disclosure is to provide a reagent for detecting methyl salicylate serving as a plant hormone released when the plant is infected with a pathogen, a methyl salicylate sensor, a method for sensing methyl salicylate using the same, and a method for detecting plant pathogen infection by the same in cultivation of plants including agricultural products.

The present inventors have intensively studied to solve the above problems. As a result, the present inventors have found that using a reagent containing a terbium compound and an amine as a receptor for methyl salicylate promotes a reaction between the terbium compound and methyl salicylate, increases fluorescence emission derived from the complex generated in association with the reaction, and enables detection of plant pathogen infection, thereby completing the present disclosure.

One aspect of the present example embodiment relates to a reagent for detecting methyl salicylate, the reagent containing a terbium compound and an amine. In addition, one aspect of the present example embodiment relates to a methyl salicylate sensor for detecting methyl salicylate, the methyl salicylate sensor including a capture unit including the reagent and used for capturing methyl salicylate.

Furthermore, one aspect of the present example embodiment relates to a method for sensing methyl salicylate by using the reagent to detect methyl salicylate, the method including
(i) reacting a terbium compound with methyl salicylate to form a complex,
(ii) applying excitation light to the complex, and
(iii) detecting fluorescence emitted by the complex.

In addition, one aspect of the present example embodiment relates to a method for detecting plant pathogen infection, the method including placing the reagent in a vicinity of a plant, and observing fluorescence emission derived from a complex formed by a reaction between a terbium compound and methyl salicylate.

According to the present disclosure, inclusion of a terbium compound and an amine as receptors for methyl salicylate in a reagent for detecting methyl salicylate increases the intensity of fluorescence emission from a complex formed by a reaction between the methyl salicylate and the terbium compound, the methyl salicylate serving as a volatile plant hormone released when a plant is infected with a pathogen, and enables detection of infection of the plant with the pathogen.

### BRIEF DESCRIPTION OF THE DRAWINGS

FIG. 1 illustrates a fluorescence spectrum curve obtained in the present disclosure and a fluorescence spectrum curve obtained in Comparative Example 1;

FIG. 2 illustrates a fluorescence spectrum curve obtained in the present disclosure;

FIG. 3 illustrates a fluorescence spectrum curve obtained in the present disclosure; and

FIG. 4 illustrates a fluorescence spectrum curve obtained in the present disclosure.

### EXAMPLE EMBODIMENT

Hereinafter, embodiments for carrying out the present disclosure will be described with reference to the drawings and the like. The example embodiments described below have technically preferable limitations for carrying out the present disclosure, but the scope of the present invention is not limited to the followings.
[1] Reagent for Detecting Methyl Salicylate

One example embodiment of the present disclosure relates to a reagent for detecting methyl salicylate, the reagent containing a terbium compound and an amine as a receptor for methyl salicylate. In the present disclosure, the term “reagent” is defined as a chemical substance used for detection or quantification of a substance by a chemical method, an experiment of synthesis of a substance, or measurement of physical properties.

The reagent also preferably contains a non-volatile ionic liquid used for effectively capturing methyl salicylate in addition to the terbium compound and the amine. In a case where the reagent contains the ionic liquid, the terbium compound and the amine are dissolved and present in the non-volatile ionic liquid, but some of the terbium compound and the amine may be precipitated. The terbium compound can similarly function as the receptor for methyl salicylate regardless of whether it is dissolved or precipitated in the non-volatile ionic liquid.

### <Receptor for Methyl Salicylate: Terbium Compound>

Examples of terbium compounds that can be used for recognizing methyl salicylate in the present disclosure include, but are not limited to, terbium compounds represented by the following General Formula (1).

Ln_{c}TbXₐY_{b} (1)

In the formula, Tb is terbium, Ln is at least one of rare earth elements other than terbium, and specific examples thereof include scandium, yttrium, lanthanum, cerium, praseodymium, neodymium, promethium, samarium, europium, gadolinium, dysprosium, holmium, erbium, thulium, ytterbium, and lutetium.

In the formula, X and Y are each independently at least one monovalent anion selected from the group consisting of RCOO⁻, halide ions and nitrate ions. R is at least one selected from the group consisting of a linear or branched alkyl group having 1 to 20 carbon atoms, a cycloalkyl group having 3 to 6 carbon atoms, and an aryl group having 6 to 14 carbon atoms, each being optionally substituted with halogen; preferably an alkyl group or a phenyl group having 1 to 6 carbon atoms, optionally substituted with a fluorine atom; and still more preferably a methyl group, an ethyl group, a propyl group, a 2-methylpropyl group, a trifluoromethyl group, a tert-butyl group, or an isobutyl group. Specific examples of RCOO⁻ preferably include at least one selected from the group consisting of an acetate ion, a trifluoroacetate ion, a propionate ion, a butyrate ion, an isobutyrate ion, a pivalate ion, a 2-methylbutyrate ion, a benzoate ion, a chloride ion, and a nitrate ion. Specific examples of the halide ion include a fluoride ion, a chloride ion, a bromide ion, and an iodide ion, and a chloride ion is preferable. Each of X and Y may be two or more kinds.

In the formula, a, b, and c are numbers satisfying 0<a, 0≤b, 0≤c≤0.05, and a+b=3+3×c.

Specific examples of the terbium compound in a case of b=c=0 among the compound represented by General Formula (1) include terbium acetate, terbium propionate, terbium butyrate, terbium isobutyrate, terbium pivalate, terbium benzoate, terbium nitrate, and terbium chloride.

Specific examples of the terbium compound in a case of b≠0 and c=0 among the compounds represented by General Formula (1) include a terbium acetate/terbium pivalate complex salt, a terbium 2-methylbutyrate/terbium pivalate complex salt, a terbium trifluoroacetate/terbium pivalate complex salt, and a terbium acetate-2-methyl/terbium butyrate complex salt. For example, the terbium acetate-terbium pivalate complex salt (Tb(CH₃COO)ₐ(t-C₄H₉COO)_{b}) can be synthesized by completely dissolving terbium acetate and terbium pivalate in methanol and conducting a heating reaction. In addition, a ratio of the counter ions (X, Y) of the complex salts can be changed by changing blending ratios of the terbium salts during the synthesis.

Specific examples of the terbium compound having b≠0 and c≠0 among the compounds represented by General Formula (1) include Ce_{0.02}Tb(CH₃COO)_{0.81}(t-C₄H₉COO)_{2.25}, Eu_{0.02}Tb(CH₃COO)_{0.81}(t-C₄H₉COO)_{2.25}, Sm_{0.02}Tb(CH₃COO)_{0.81}(t-C₄H₉COO)_{2.25}, Dy_{0.02}Tb(CH₃COO)_{0.81}(t-C₄H₉COO)_{2.25}, Gd_{0.02}Tb(CH₃COO)_{0.81}(t-C₄H₉COO)_{2.25}, Yb_{0.02}Tb(CH₃COO)_{0.81}(t-C₄H₉COO)_{2.25}, Ce_{0.01}Tb(CH₃COO)_{0.78}(t-C₄H₉COO)_{2.25}, Ce_{0.015}Tb(CH₃COO)_{0.795}(t-C₄H₉COO)_{2.25}, Ce_{0.025}Tb(CH₃COO)_{0.825}(t-C₄H₉COO)_{2.25}, Ce_{0.04}Tb(CH₃COO)_{0.87}(t-C₄H₉COO)_{2.25}, Ce_{0.05}Tb(CH₃COO)_{0.9}(t-C₄H₉COO)_{2.25}, Ce_{0.06}Tb(CH₃COO)_{0.93}(t-C₄H₉COO)_{2.25}, Ce_{0.02}Tb(CH₃COO)_{3.06}, Ce_{0.04}Tb(CH₃COO)_{3.12}, Yb_{0.15}Tb(CH₃COO)_{0.795}(t-C₄H₉COO)_{2.25}, Yb_{0.025}Tb(CH₃COO)_{0.825}(t-C₄H₉COO)_{2.25}, and Yb_{0.04}Tb(CH₃COO)_{0.87}(t-C₄H₉COO)_{2.25}. For example, Ce_{c}Tb(CH₃COO)ₐ(t-C₄H₉COO)_{b} can be synthesized by dissolving terbium acetate, terbium pivalate, and any amount of cerium acetate by mole with respect to terbium (Tb) in methanol and conducting a heating reaction. In addition, the ratio of the counter ions (X, Y) and the doping amount (c) of cerium can be changed by changing the ratio of the two terbium salts (terbium acetate and terbium pivalate) during the synthesis and the molar ratio of cerium acetate.

In a case where a rare earth element (Ln) other than terbium is doped, c is preferably 0.015 to 0.05. In a case where two or more rare earth elements (Ln) other than terbium are doped, the total c is also preferably 0.015 to 0.05. In a case where c is within this range, the effect of increasing the intensity of fluorescence emission derived from the complex formed by the reaction with methyl salicylate is sufficiently exhibited.

These terbium compounds can selectively recognize methyl salicylate by each reacting with methyl salicylate to form a complex.

When excited by UV, the produced methyl salicylate/terbium complex emits fluorescence specific to a terbium complex. In a case where only the terbium compound is used, the intensity of fluorescence emission is small even under irradiation with UV light, and the fluorescence emission is thus not observed. In addition, the terbium compound can selectively recognize methyl salicylate because the terbium compound does not react with and thus does not recognize other plant hormones other than methyl salicylate, for example, methyl jasmonate.

### <Amine>

In order to promote the reaction between the terbium compound and methyl salicylate, the reagent of the present disclosure contains an amine with basicity. Examples of the amine that can be used in the present disclosure include amines having 6to 30 carbon atoms, and specific examples thereof include, but are not limited only to, triethylamine, tripropylamine, tributylamine, triisobutylamine, triamylamine, triisoamylamine, trihexylamine, triheptylamine, tri-n-octylamine, tris(2-ethylhexylamine), tridecylamine, tribenzylamine, triphenylamine, and N,N-diethylaniline. As illustrated in the following formula, these amines accelerate the reaction between methyl salicylate and the terbium compound by interacting with a hydrogen atom of a phenolic hydroxyl group of methyl salicylate during the reaction between methyl salicylate and the terbium compound, resulting in an increase in fluorescence intensity. A molar ratio of the amine to the terbium compound is preferably 0.5:1 to 10:1, more preferably 2:1 to 7:1, and particularly preferably 3:1 to 5:1. In a case where the molar ratio of the amine to the terbium compound is less than 0.5:1, the effect of accelerating the reaction with methyl salicylate may be reduced, and the effect of improving the detection sensitivity may not be obtained, and in a case where the molar ratio of the amine to the terbium compound is more than 10:1, the terbium compound may be difficult to form a complex with methyl salicylate.

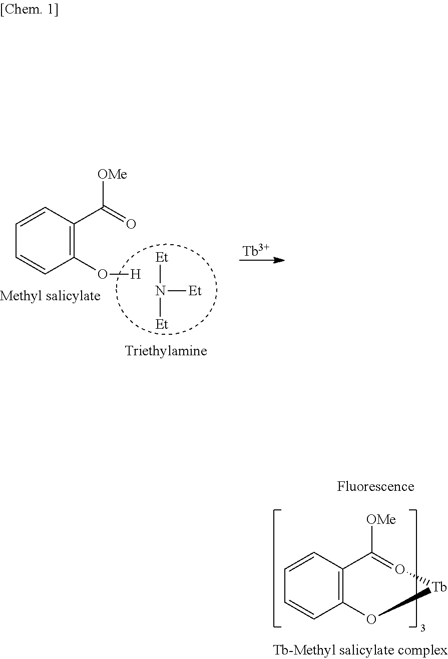

### Reaction of Methyl Salicylate with Terbium Compound

### <Non-Volatile Ionic Liquid>

The reagent of the present disclosure preferably contains a non-volatile ionic liquid in order to effectively capture methyl salicylate. Examples of the non-volatile ionic liquid that can be used in the present disclosure include an imidazolium salt, a phosphonium salt, a pyridinium salt, an ammonium salt, a piperidinium salt, and a pyrrolidinium salt. Specific examples thereof include, but are not limited to, 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butyl-3-methylimidazolium dibutyl phosphate, tetrabutyl ammonium acetate, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium dicyanamide.

A weight ratio of the ionic liquid to the terbium compound is preferably 2:1 to 40:1, more preferably 3:1 to 30:1, and particularly preferably 4:1 to 20:1. In a case where the weight ratio of the ionic liquid to the terbium compound is less than 2:1, the effect of improving the detection sensitivity for methyl salicylate may not be obtained, and in a case where the weight ratio of the ionic liquid to the terbium compound is more than 40:1, the terbium compound may be difficult to form a complex with methyl salicylate.

The reagent for detecting methyl salicylate of the present disclosure may optionally contain another solvent within the scope that does not impair the effects of the present disclosure. Examples of the solvent that can be used include, but are not limited to, dimethylsulfoxide, methanol, ethanol, water, N,N-dimethylformamide, tetrahydrofuran, acetone, acetonitrile, and 1,4-dioxane.

### [2] Methyl Salicylate Sensor

One example embodiment of the present disclosure relates to a methyl salicylate sensor for detecting methyl salicylate, the methyl salicylate sensor including a capture unit including the reagent and used for capturing methyl salicylate. Preferably, the methyl salicylate sensor includes a detection unit that detects whether methyl salicylate is captured by the capture unit.

### (1) Capture Unit

The capture unit of the methyl salicylate sensor of the present disclosure includes a reagent that contains a terbium compound serving as a receptor selectively capturing methyl salicylate, and an amine accelerating a reaction between the terbium compound and methyl salicylate. In the capture unit, the reagent is preferably contained in a medium.

### <Medium>

Examples of the medium containing the terbium compound and the amine of the present disclosure may include, but are not limited to, paper or glass fiber, a resin (for example, polymethyl methacrylate, polyethylene, polypropylene, polyvinyl chloride, polystyrene, nylon resin, polyamide, polycarbonate, polyethylene terephthalate, polybutylene terephthalate, or polyphenylene oxide), or a water soluble polymer (cellulose-based polymer, agarose, starch-based polymer, sodium alginate, acrylic acid-based polymer, acrylamide-based polymer, polyvinyl alcohol, polyethylene oxide, polyvinyl pyrrolidone, and the like).

For example, in a case where paper is used as the medium, a medium containing the terbium compound and the amine is obtained by dissolving the terbium compound and the amine in a solvent, impregnating paper (for example, filter paper) with the obtained solution, and then drying the paper at a temperature ranging from room temperature to 60° C. to remove the solvent. After drying, the solvent is evaporated and removed, but may remain. It is preferable to use a solvent because the terbium compound is easily impregnated into the medium and the concentration of the terbium compound is easily adjusted.

Examples of the solvent in which the terbium compound is dissolved include, but are not limited to, dimethylsulfoxide, methanol, ethanol, water, N,N-dimethylformamide, tetrahydrofuran, acetone, acetonitrile, and 1,4-dioxane.

It is preferable to add the aforementioned non-volatile ionic liquid to the solvent in order to effectively capture methyl salicylate. In a case of using the non-volatile ionic liquid, after the solvent is removed, the terbium compound is dissolved and present in the non-volatile ionic liquid, but a part of the terbium compound may be precipitated. The terbium compound can similarly function as the receptor for methyl salicylate regardless of whether it is dissolved or precipitated in the non-volatile ionic liquid.

In addition, a ratio of the ionic liquid to the solvent may be appropriately set with respect to the medium for impregnation. In a case where the ratio of the ionic liquid to the solvent decreases, the amount of the ionic liquid in the medium after drying is reduced, and the effect of improving the detection sensitivity may be reduced. In contrast, in a case where the ratio of the ionic liquid to the solvent increases, a disadvantage that impregnation in the medium is difficult may occur because a viscosity of the ionic liquid increases. Therefore, the ratio of the ionic liquid to the solvent is appropriately set with respect to the medium. For example, in a case where the filter paper is impregnated with the ionic liquid, the ratio of the ionic liquid to the solvent is preferably 5% to 50% by weight, and more preferably 10% to 30% by weight.

### (2) Detection Unit

The detection unit of the methyl salicylate sensor of the present disclosure is configured in such a way as to optically detect whether methyl salicylate is captured by the capture unit. The detection unit may be configured as a separate device instead of an integrated device with the capture unit. In one aspect of the present disclosure, the optical detection unit includes an excitation light source (light emitting unit) and a detection element (light receiving unit of fluorescence) in order to detect fluorescence emission from the complex produced by reacting the terbium compound with methyl salicylate, and can detect methyl salicylate and/or measure the concentration of methyl salicylate based on the observed change in fluorescence intensity.

In one aspect of the present disclosure, the detection unit may include a computer that executes a computer program to process detection and/or concentration measurement of methyl salicylate. Such a computer program may be, for example, a computer program that causes a computer to execute the steps of (i) receiving a signal from an optical detection element, (ii) analyzing the received signal to determine the presence or absence and/or concentration of methyl salicylate, and (iii) outputting an analysis result.

In one aspect of the present disclosure, the analysis of the received signal may include comparing the received signal to a predetermined reference value to determine the presence or absence and/or concentration of methyl salicylate, for example. In addition, in one aspect of the present disclosure, the analysis result may be output to, for example, a display device connected to the sensor, another device connected via a network, or the like.

In one aspect of the present disclosure, the methyl salicylate sensor of the present disclosure senses methyl salicylate serving as a plant hormone released when a crop is infected with a pathogen. Therefore, the methyl salicylate sensor of the present disclosure may be used as a sensor for detecting pathogen infection of plants including crops.

### [3] Method for Sensing Methyl Salicylate

One example embodiment of the present disclosure relates to a method for sensing methyl salicylate by using the above-described reagent or methyl salicylate sensor based on the phenomenon of fluorescence emission from a complex obtained by reacting a terbium compound with methyl salicylate, the method including
(i) reacting a terbium compound with methyl salicylate to form a complex,
(ii) applying excitation light to the complex, and
(iii) detecting fluorescence emitted by the complex.

While fluorescence emission is hardly exhibited only with the terbium compound, the complex produced by the reaction between the terbium compound and methyl salicylate comes to exhibit fluorescence emission. Utilizing this phenomenon enables detection of methyl salicylate.

In one aspect of the present disclosure, a suitable wavelength is selected within a range of 300 to 400 nm as an excitation wavelength. Furthermore, in one aspect of the present disclosure, it is also possible to perform comparing the intensity of the detected fluorescence to a predetermined reference value to determine the concentration of methyl salicylate.

### [4] Method for Detecting Plant Pathogen Infection

One example embodiment of the present disclosure relates to a method for detecting plant pathogen infection, the method including placing the reagent or the methyl salicylate sensor in the vicinity of a plant, and observing fluorescence emission derived from a complex formed by a reaction between a terbium compound and methyl salicylate.

Examples of the plant that may be a monitoring target include, but are not limited to, cucumber, watermelon, tomato, eggplant, bell pepper, paprika, shishito pepper, melon, Chinese cabbage, cabbage, radish, lettuce, green onion, broccoli, onion, garlic, yam, asparagus, carrot, potato, celery, tobacco, rice, and strawberry.

Examples of the disease that may be detected include, but are not limited to, ring spot disease, leaf spot, Corynespora target spot, leaf mold, fusarium wilt, root rot wilt, Verticillium wilt, brown root rot, gray phytophthora rot, root rot, black dot root rot, southern blight, damping off, brown leaf spot, downy mildew, powdery mildew, gray mold, anthracnose, scab, Sclerotinia rot, gummy stem blight, leaf spot, blight, mosaic disease, spotted wilt, yellow leaf curl, bacterial wilt, bacterial soft rot, bacterial canker, pith necrosis, bacterial black spot, and bacterial leaf spot, and examples of the pathogen infection that may be detected include, but are not limited to, infections caused by the causative microorganisms of the above-described diseases.

In the context of the present disclosure, in a case of referring to “installed in the vicinity of a plant”, examples of the term “vicinity” include, but are not limited to, a distance of within 2 m, within 1 m, within 75 cm, within 50 cm, within 40 cm, within 30 cm, within 20 cm, within 10 cm, or within 5 cm from the plant to be monitored, and an appropriate distance is appropriately selected in view of various factors. A person skilled in the art can appropriately set a position where the sensor is installed in consideration of various conditions.

Furthermore, one example embodiment of the present disclosure relates to the use of the reagent or methyl salicylate sensor in the detection of plant pathogen infection.

In addition, one example embodiment of the present disclosure also relates to the use of the terbium compound and the amine in manufacturing the reagent or methyl salicylate sensor.

### EXAMPLES

Hereinafter, the present disclosure is described in more detail with reference to examples, but the present invention is not limited to such examples.

### Synthesis Example 1

Terbium Acetate (Tb(CH₃COO)₃:TbA)-Terbium Pivalate (Tb(t-C₄H₉COO)₃:TbPv) Complex Salt Doped With 2% Cerium (Ce) [Ce_{0.02}Tb(CH₃COO)_{0.81}(C₄H₉COO)_{2.25}:0.02CeA-0.25TbA-0.75TbPv]

In 120 ml of methanol are dissolved 0.2 g of terbium acetate tetrahydrate, 0.6797 g of terbium pivalate, and 0.0131 g of cerium acetate monohydrate, and the resultant solution is heated and refluxed for 5 hours. After the mixture was left to cool, methanol was distilled off, and the precipitated white crystals were vacuum-dried to obtain 0.733 g of a desired terbium compound doped with 2% cerium.

### Fluorescence Emission Behavior Associated With Reaction With Methyl Salicylate

### Example 1

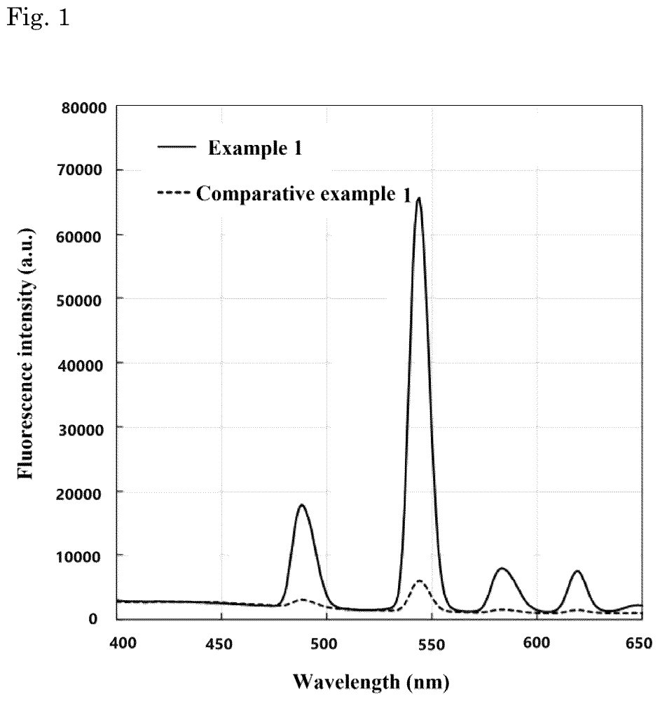

In 1.6 ml of dimethyl sulfoxide (DMSO) was dissolved 0.0437 g of the terbium acetate-terbium pivalate complex salt doped with 2% cerium (0.02CeA-0.25TbA-0.75TbPv) obtained in Synthesis Example 1, 0.07 ml of triethylamine (NEt₃) (5 molar equivalents with respect to terbium) and 0.4 ml of 1-ethyl-1-methylimidazolium acetate (EMImAc) were added thereto, and 0.2 ml of the solution was added dropwise to a circular filter paper (Φ40 mm) and dried to volatilize DMSO, thereby obtaining a filter paper containing 0.02CeA-0.25TbA-0.75TbPv, NEt₃, and EMImAc. The obtained filter paper was sandwiched between two glass plates (40 mm square, 2 mm thickness, with ¼ inch hole in the center) and secured with clips, a Teflon tube was inserted into the ¼ hole, and methyl salicylate at a concentration of 0.01 ug/L was passed through Permeater PD-1B-2 (manufactured by GASTEC CORPORATION) with nitrogen as a carrier gas. The filter paper was taken out after exposure for 1 hour, and a fluorescence spectrum at an excitation wavelength of 365 nm was measured. The fluorescence spectrum obtained in Example 1 is illustrated by the solid line in FIG. 1.

### Comparative Example 1

A fluorescence spectrum at an excitation wavelength of 365 nm was measured in the same manner as in Example 1 except that triethylamine (NEt₃) was not used. The fluorescence spectrum obtained in Comparative Example 1 is indicated by the broken line in FIG. 1.

As illustrated in FIG. 1, the fluorescence intensity (Example 1) at a wavelength of 544 nm in the fluorescence spectrum containing triethylamine was increased to about 11times the fluorescence intensity (Comparative Example 1) at a wavelength of 544 nm in the fluorescence spectrum containing no triethylamine. As described above, it was demonstrated that methyl salicylate in the gas phase at a concentration of 0.01 ug/L could be easily sensed since triethylamine was contained.

### Example 2

An evaluation was carried out in the same manner as in Example 1 except that 0.0453 g of terbium nitrate hexahydrate was used in place of the terbium acetate-terbium pivalate complex salt doped with 2% cerium (0.02CeA-0.25TbA-0.75TbPv).

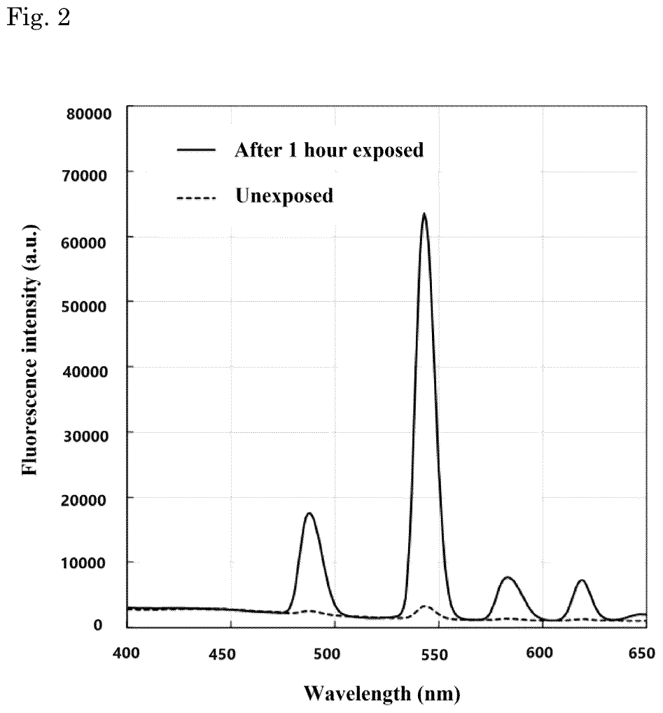

FIG. 2 illustrates a fluorescence spectrum after exposure for 1 hour (solid line) and a fluorescence spectrum without exposure (broken line). The fluorescence intensity at a wavelength of 544 nm in the fluorescence spectrum after exposure for 1 hour was increased to about 33 times the fluorescence intensity at a wavelength of 544 nm in the fluorescence spectrum without exposure. As described above, it was demonstrated that methyl salicylate in the gas phase at a concentration of 0.01 ug/L could be sensed by using the reagent containing the terbium compound and the amine.

### Example 3

An evaluation was carried out in the same manner as in Example 2 except that 0.072 ml of triisobutylamine (5 molar equivalents with respect to terbium) was used in place of triethylamine.

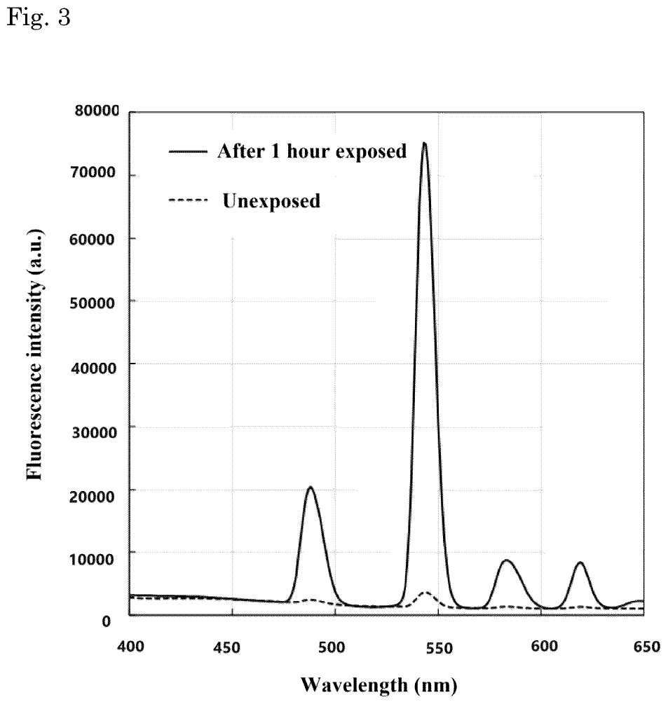

FIG. 3 illustrates a fluorescence spectrum after exposure for 1 hour (solid line) and a fluorescence spectrum without exposure (broken line). The fluorescence intensity at a wavelength of 544 nm in the fluorescence spectrum after exposure for 1 hour was increased to about 32 times the fluorescence intensity at a wavelength of 544 nm in the fluorescence spectrum without exposure. As described above, it was demonstrated that methyl salicylate in the gas phase at a concentration of 0.01 ug/L could be sensed by using the reagent containing the terbium compound and the amine.

### Example 4

An evaluation was carried out in the same manner as in Example 2 except that 0.13 ml of tris(2-ethylhexyl)amine (3 molar equivalents with respect to terbium) was used in place of triethylamine.

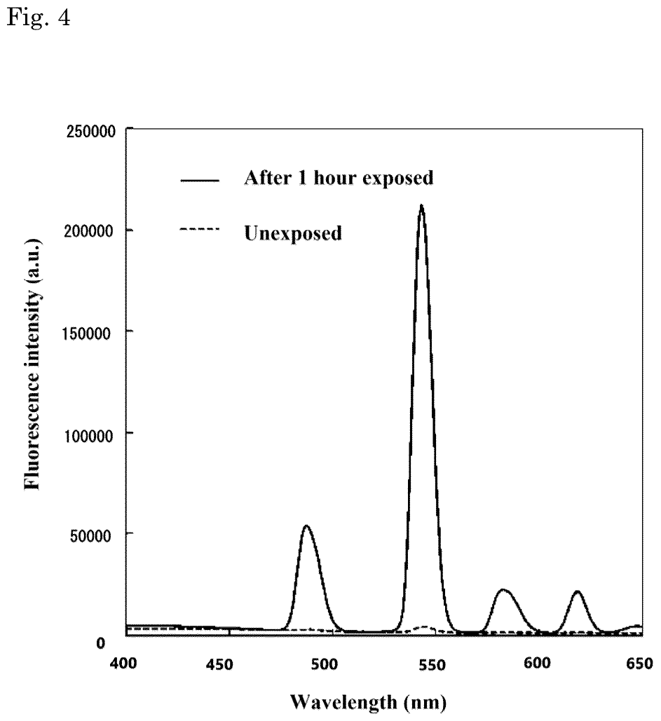

FIG. 4 illustrates a fluorescence spectrum after exposure for 1 hour (solid line) and a fluorescence spectrum without exposure (broken line). The fluorescence intensity at a wavelength of 544 nm in the fluorescence spectrum after exposure for 1 hour was increased to about 75 times the fluorescence intensity at a wavelength of 544 nm in the fluorescence spectrum without exposure. As described above, it was demonstrated that methyl salicylate in the gas phase at a concentration of 0.01 ug/L could be sensed by using the reagent containing the terbium compound and the amine.

### Example 5

An evaluation was carried out in the same manner as in Example 1 except that 0.0408 g of terbium acetate tetrahydrate was used in place of the terbium acetate-terbium pivalate complex salt doped with 2% cerium (0.02CeA-0.25TbA-0.75TbPv). The fluorescence intensity at a wavelength of 544 nm in the fluorescence spectrum after exposure for 1 hour was increased to about 8 times the fluorescence intensity without exposure. As described above, it was demonstrated that methyl salicylate in the gas phase at a concentration of 0.01 ug/L could be sensed by using the reagent containing the terbium compound and the amine.

### Example 6

An evaluation was carried out in the same manner as in Example 1 except that 0.0373 g of terbium chloride hexahydrate was used in place of the terbium acetate-terbium pivalate complex salt doped with 2% cerium (0.02CeA-0.25TbA-0.75TbPv). The fluorescence intensity at a wavelength of 544 nm in the fluorescence spectrum after exposure for 1 hour was increased to about 23 times the fluorescence intensity without exposure. As described above, it was demonstrated that methyl salicylate in the gas phase at a concentration of 0.01 ug/L could be sensed by using the reagent containing the terbium compound and the amine.

### Example 7

An evaluation was carried out in the same manner as in Example 1 except that 0.0462 g of terbium pivalate was used in place of the terbium acetate-terbium pivalate complex salt doped with 2% cerium (0.02CeA-0.25TbA-0.75TbPv). The fluorescence intensity at a wavelength of 544 nm in the fluorescence spectrum after exposure for 1 hour was increased to about 15 times the fluorescence intensity without exposure. As described above, it was demonstrated that methyl salicylate in the gas phase at a concentration of 0.01 ug/L could be sensed by using the reagent containing the terbium compound and the amine.

### Example 8

An evaluation was carried out in the same manner as in Example 1 except that the addition amount of triethylamine was changed to 0.042 ml (3 molar equivalents with respect to terbium). The fluorescence intensity at a wavelength of 544 nm in the fluorescence spectrum after exposure for 1 hour was increased to about 13 times the fluorescence intensity without exposure. As described above, it was demonstrated that methyl salicylate in the gas phase at a concentration of 0.01 ug/L could be easily sensed since triethylamine was contained.

Since the methyl salicylate sensor disclosed herein for detecting methyl salicylate serving as a plant hormone includes the capture unit including the medium containing the terbium compound and the amine, and used for efficiently capturing methyl salicylate to form the complex, leading to expression of fluorescence emission, it is possible to selectively detect methyl salicylate serving as a plant hormone released when a plant is infected with a pathogen. Then, the plant pathogen infection can be detected by using the methyl salicylate sensor, and specifically, as a sensor capable of detecting pathogen infection of agricultural crops, the methyl salicylate sensor can be used as a new sensor for agricultural ICT in protected horticulture such as greenhouse.

While the present disclosure has been described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

### (Supplementary Note 1)

A reagent for detecting methyl salicylate, the reagent containing: a terbium compound; and an amine.

### (Supplementary Note 2)

The reagent according to Supplementary Note 1, in which the amine has 6 to 30 carbon atoms.

### (Supplementary Note 3)

The reagent according to Supplementary Note 1 or 2, in which the amine is at least one selected from the group consisting of triethylamine, tripropylamine, tributylamine, triisobutylamine, triamylamine, triisoamylamine, trihexylamine, triheptylamine, tri-n-octylamine, tris(2-ethylhexylamine), tridecylamine, tribenzylamine, triphenylamine, and N,N-diethylaniline.

### (Supplementary Note 4)

The reagent according to any of preceding Supplementary Notes, in which the terbium compound is at least one compound represented by General Formula (1):

Ln_{c}TbXₐY_{b} (1)
(in formula, Tb is terbium, Ln is at least one of rare earth elements other than terbium, X and Y are each independently at least one monovalent anion selected from the group consisting of RCOO⁻, a halide ion, and a nitrate ion, R is at least one selected from the group consisting of a linear or branched alkyl group having 1 to 20 carbon atoms, a cycloalkyl group having 3 to 6 carbon atoms, and an aryl group having 6 to 14 carbon atoms, each being optionally substituted with halogen, and a, b, and c are numbers satisfying 0<a, 0≤b, 0≤c≤0.05, and a+b=3+3×c).

### (Supplementary Note 5)

The reagent according to any of preceding Supplementary Notes, in which a molar ratio of the amine to the terbium compound is 0.5:1 to 10:1.

### (Supplementary Note 6)

The reagent according to any of preceding Supplementary Notes, in which the reagent for detecting methyl salicylate further contains at least one non-volatile ionic liquid selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium dicyanamide.

### (Supplementary Note 7)

The reagent according to any of Supplementary Notes 4 to 6, in which X and Y are each independently at least one selected from the group consisting of an acetate ion, a trifluoroacetate ion, a propionate ion, a butyrate ion, an isobutyrate ion, a pivalate ion, a 2-methylbutyrate ion, a benzoate ion, a chloride ion, and a nitrate ion.

### (Supplementary Note 8)

The reagent according to any of Supplementary Notes 4 to 8, in which the compound represented by General Formula (1) is at least one selected from the group consisting of terbium acetate, terbium propionate, terbium butyrate, terbium isobutyrate, terbium pivalate, terbium benzoate, terbium nitrate, and terbium chloride.

### (Supplementary Note 9)

The reagent according to any of Supplementary Notes 4 to 7, in which c=0.

### (Supplementary Note 10)

The reagent according to any of Supplementary Notes 4 to 7, in which 0.015≤c≤0.05.

### (Supplementary Note 11)

The reagent according to any of Supplementary Notes 4 to 7, and 10, in which Ln is at least one selected from the group consisting of scandium, yttrium, lanthanum, cerium, praseodymium, neodymium, promethium, samarium, europium, gadolinium, dysprosium, holmium, erbium, thulium, ytterbium, and lutetium.

### (Supplementary Note 12)

The reagent according to any of preceding Supplementary Notes, further containing a non-volatile ionic liquid.

### (Supplementary Note 13)

The reagent according to Supplementary Note 12, in which the non-volatile ionic liquid is at least one selected from the group consisting of an imidazolium salt, a phosphonium salt, a pyridinium salt, an ammonium salt, a piperidinium salt, and a pyrrolidinium salt.

### (Supplementary Note 14)

The reagent according to Supplementary Note 12, in which the non-volatile ionic liquid is at least one selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butyl-3-methylimidazolium dibutyl phosphate, tetrabutyl ammonium acetate, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium dicyanamide.

### (Supplementary Note 15)

The reagent according to any of Supplementary Notes 12 to 14, in which a weight ratio of the non-volatile ionic liquid to the terbium compound is 2:1 to 40:1.

### (Supplementary Note 16)

A methyl salicylate sensor for detecting methyl salicylate, the methyl salicylate sensor including:
a capture unit including the reagent according to any of preceding Supplementary Notes and used for capturing methyl salicylate.

### (Supplementary Note 17)

The methyl salicylate sensor according to Supplementary Note 16, in which the capture unit includes a medium containing the reagent.

### (Supplementary Note 18)

The methyl salicylate sensor according to Supplementary Note 16 or 17, further comprising:
a detection unit configured to detect whether methyl salicylate is captured by the capture unit.

### (Supplementary Note 19)

The methyl salicylate sensor according to any of Supplementary Notes 16 to 18, in which the medium is a paper, a glass fiber, a resin, or a water soluble polymer.

### (Supplementary Note 20)

The methyl salicylate sensor according to any of Supplementary Notes 16 to 19, in which the detection unit includes a computer executing a computer program to process detection and/or concentration measurement of methyl salicylate.

### (Supplementary Note 21)

A method for sensing methyl salicylate by using the reagent according to any of Supplementary Notes 1 to 15 to recognize methyl salicylate, the method including:
(i) reacting a terbium compound with methyl salicylate to form a complex;
(ii) applying excitation light to the complex; and
(iii) detecting fluorescence emitted by the complex.

### (Supplementary Note 22)

A method for sensing methyl salicylate by using the methyl salicylate sensor according to any of Supplementary Notes 16 to 19 to recognize methyl salicylate, the method including:
(i) reacting a terbium compound with methyl salicylate to form a complex;
(ii) applying excitation light to the complex; and
(iii) detecting fluorescence emitted by the complex.

### (Supplementary Note 23)

A method for detecting plant pathogen infection, the method including: placing the reagent according to any of Supplementary Notes 1 to 15 in a vicinity of a plant; and observing fluorescence emission derived from a complex formed by a reaction between a terbium compound and methyl salicylate.

### (Supplementary Note 24)

A method for detecting plant pathogen infection, the method including: placing the methyl salicylate sensor according to any of Supplementary Notes 16 to 19 in a vicinity of a plant; and observing fluorescence emission derived from a complex formed by a reaction between a terbium compound and methyl salicylate.

1. A reagent for detecting methyl salicylate, the reagent comprising: a terbium compound; and an amine.

2. The reagent according to claim 1, wherein the amine has 6 to 30 carbon atoms.

3. The reagent according to claim 1, wherein the amine is at least one selected from the group consisting of triethylamine, tripropylamine, tributylamine, triisobutylamine, triamylamine, triisoamylamine, trihexylamine, triheptylamine, tri-n-octylamine, tris(2-ethylhexylamine), tridecylamine, tribenzylamine, triphenylamine, and N,N-diethylaniline.

4. The reagent according to claim 1, wherein
the terbium compound is at least one compound represented by General Formula (1):
Ln_{c}TbXₐY_{b} (1)
wherein Tb is terbium, Ln is at least one of rare earth elements other than terbium, X and Y are each independently at least one monovalent anion selected from the group consisting of RCOO⁻, a halide ion, and a nitrate ion, R is at least one selected from the group consisting of a linear or branched alkyl group having 1 to 20 carbon atoms, a cycloalkyl group having 3 to 6 carbon atoms, and an aryl group having 6 to 14 carbon atoms, each being optionally substituted with halogen, and a, b, and c are numbers satisfying 0<a, 0≤b, 0≤c≤0.05, and a+b=3+3×c.

5. The reagent according to claim 1, wherein a molar ratio of the amine to the terbium compound is 0.5:1 to 10:1.

6. The reagent according to claim 1, wherein the reagent for detecting methyl salicylate further comprises at least one non-volatile ionic liquid selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butylpyridinium bis (trifluoromethanesulfonyl) imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium dicyanamide.

7. A methyl salicylate sensor configured to detect methyl salicylate, the methyl salicylate sensor comprising:
a capture unit including the reagent according to claim 1 and configured to capture methyl salicylate.

8. The methyl salicylate sensor according to claim 7, wherein the capture unit includes a medium containing the reagent.

9. The methyl salicylate sensor according to claim 7, further comprising:
a detection unit configured to detect whether methyl salicylate is captured by the capture unit.

10. A method for sensing methyl salicylate by using the reagent according to claim 1 to recognize methyl salicylate, the method comprising:
(i) reacting a terbium compound with methyl salicylate to form a complex;
(ii) applying excitation light to the complex; and
(iii) detecting fluorescence emitted by the complex.

11. A method for detecting plant pathogen infection, the method comprising: placing the reagent according to claim 1 in a vicinity of a plant; and observing fluorescence emission derived from a complex formed by a reaction between a terbium compound and methyl salicylate.

12. The reagent according to claim 4, in which X and Y are each independently at least one selected from the group consisting of an acetate ion, a trifluoroacetate ion, a propionate ion, a butyrate ion, an isobutyrate ion, a pivalate ion, a 2-methylbutyrate ion, a benzoate ion, a chloride ion, and a nitrate ion.

13. The reagent according to claim 4, in which the compound represented by General Formula (1) is at least one selected from the group consisting of terbium acetate, terbium propionate, terbium butyrate, terbium isobutyrate, terbium pivalate, terbium benzoate, terbium nitrate, and terbium chloride.

14. The reagent according to claim 4, in which c=0.

15. The reagent according to claim 4, in which 0.015≤c≤0.05.

16. The reagent according to claim 4, in which Ln is at least one selected from the group consisting of scandium, yttrium, lanthanum, cerium, praseodymium, neodymium, promethium, samarium, europium, gadolinium, dysprosium, holmium, erbium, thulium, ytterbium, and lutetium.

17. The reagent according to claim 1, further comprising a non-volatile ionic liquid.

18. The reagent according to claim 17, in which the non-volatile ionic liquid is at least one selected from the group consisting of an imidazolium salt, a phosphonium salt, a pyridinium salt, an ammonium salt, a piperidinium salt, and a pyrrolidinium salt.

19. The reagent according to claim 17, in which the non-volatile ionic liquid is at least one selected from the group consisting of 1-ethyl-3-methylimidazolium acetate, 1-butyl-3-methylimidazolium acetate, 1-ethyl-3-methylimidazolium tetrafluoroborate, 1-butyl-1-methylpyrrolidinium bis(fluorosulfonyl)imide, 1-butyl-3-methylimidazolium dibutyl phosphate, tetrabutyl ammonium acetate, 1-butylpyridinium bis(trifluoromethanesulfonyl)imide, 1-methyl-1-propylpiperidinium bis(fluorosulfonyl)imide, 1-ethyl-3-methylimidazolium bis(trifluoromethanesulfonyl)imide, and 1-butyl-3-methylimidazolium dicyanamide.

20. The reagent according to claim 17, in which a weight ratio of the non-volatile ionic liquid to the terbium compound is 2:1 to 40:1.
